(19) United States
(12) Patent Application Publication
(10) Pub. No.: US 2023/0250781 A1
(43) Pub. Date: Aug. 10, 2023

(54) METHOD AND SYSTEM FOR OPERATING A ROBOTIC DEVICE

(71) Applicant: Tombot, Inc., Santa Clarita, CA (US)

(72) Inventors: Thomas Edward Stevens, Westlake Village, CA (US); Henry Peter Schorz, Santa Clarita, CA (US); Jesse Michael Schorz, Santa Clarita, CA (US)

(21) Appl. No.: 18/297,574

(22) Filed: Apr. 7, 2023

Related U.S. Application Data
(63) Continuation of application No. 16/853,311, filed on Apr. 20, 2020, now abandoned.
(60) Provisional application No. 62/836,530, filed on Apr. 19, 2019.

(51) Int. Cl. F02M 25/12; C25B 1/02; C25B 9/00

(52) U.S. Cl. CPC F02M 25/12; C25B 1/02; C25B 9/00; F02B 43/12

(57) ABSTRACT

Embodiments of a method (e.g., for operating a robotic device such as a dog device, etc.) can include: receiving one or more inputs (e.g., sensor input data, etc.) at a dog device (e.g., at one or more sensors of the dog device; a robotic dog device; etc.) from one or more users and/or other suitable entities (e.g., additional dog devices; etc.); determining one or more events (and/or a lack of one or more events), such as based on the one or more inputs (and/or a lack of one or more inputs); processing (e.g., determining, implementing, etc.) one or more scenes based on the one or more events (and/or lack of one or more events); and/or performing one or more output actions with the dog device, based on the one or more scenes (e.g., individual scenes; scene flows; etc.).

### CROSS-REFERENCE TO RELATED APPLICATIONS

This application is a continuation of U.S. application Ser. No. 16/853,311, filed on 20 Apr. 2020, which claims the benefit of U.S. Provisional Application Ser. No. 62/836,530, filed on 19 Apr. 2019, each of which is incorporated herein in its entirety by this reference.

### TECHNICAL FIELD

The disclosure generally relates to robotics.

### BRIEF DESCRIPTION OF THE FIGURES

FIG. 1 includes a schematic representation of an embodiment of a method;

FIG. 2 includes a graphic representation of an embodiment of a method;

FIG. 3 includes a specific example of a distribution of functionality across components of a computing system;

FIG. 4 includes a specific example of a main flow;

FIG. 5 includes a specific example of an event-related flow;

FIG. 6 includes a specific example of processes performed at initialization of a dog device;

FIG. 7 includes a specific example of a scene flow;

FIG. 8 includes a specific example of a scene flow;

FIG. 9 includes a specific example of events and corresponding scene types;

FIG. 10 includes a specific example of statuses indicated by a physical input receiving component;

FIG. 11 includes a specific example flow associated with mechanical actuators;

FIG. 12 includes a specific example of an embodiment of a system;

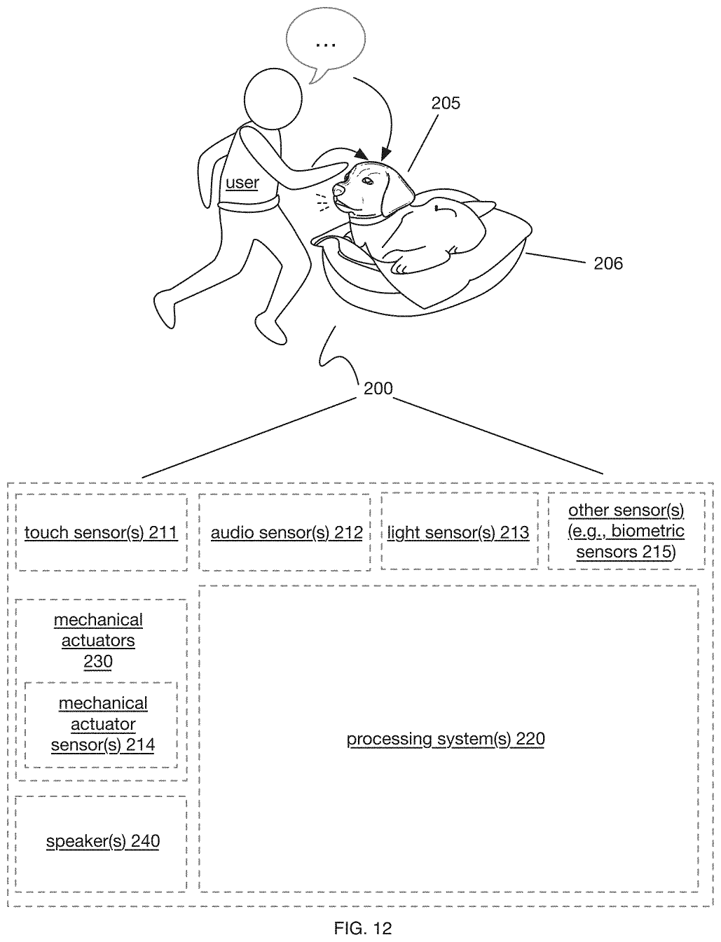

FIG. 13 includes a specific example associated with light sensors.

### DESCRIPTION OF THE EMBODIMENTS

The following description of the embodiments (e.g., including variations of embodiments, examples of embodiments, specific examples of embodiments, other suitable variants, etc.) is not intended to be limited to these embodiments, but rather to enable any person skilled in the art to make and use.

### 1. Overview

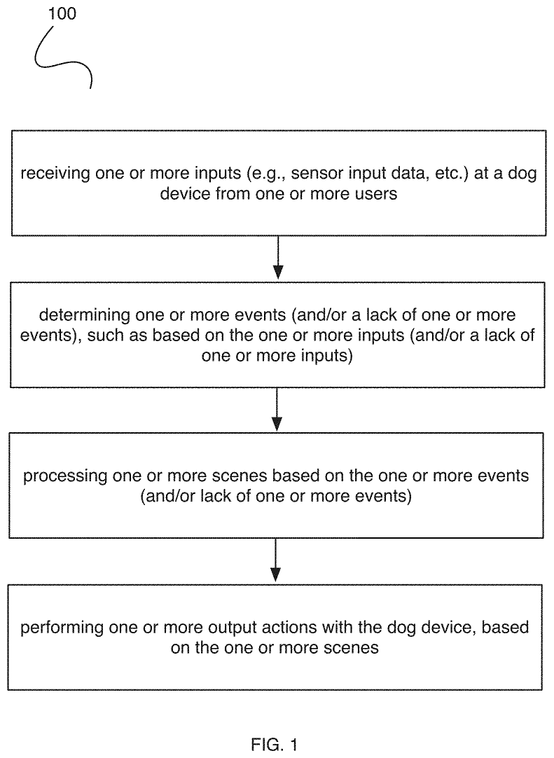

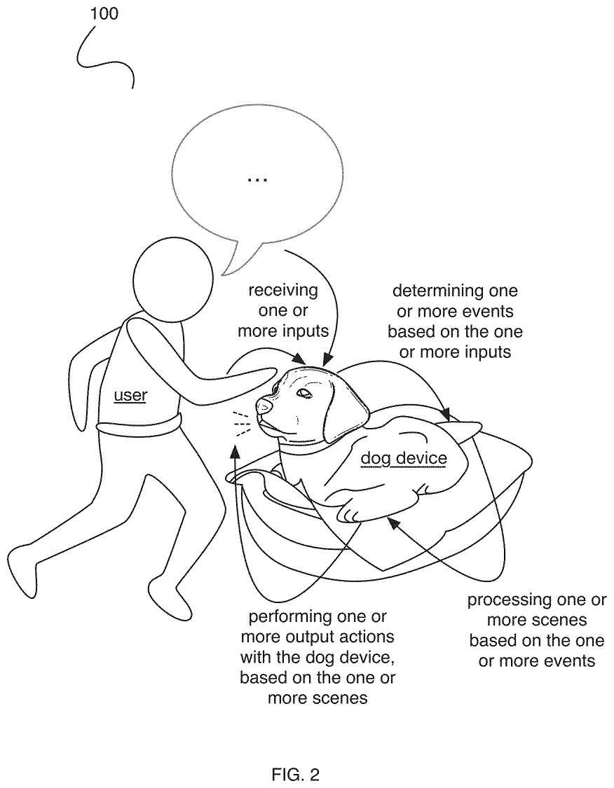

As shown in FIG. 1-2, embodiments of a method 100 (e.g., for operating a robotic device such as a dog device, etc.) can include: receiving one or more inputs (e.g., sensor input data, etc.) at a dog device (e.g., at one or more sensors of the dog device; a robotic dog device; etc.) (and/or or any suitable robotic device) from one or more users and/or other suitable entities (e.g., additional dog devices; etc.); determining one or more events (and/or a lack of one or more events), such as based on the one or more inputs (and/or a lack of one or more inputs); processing (e.g., determining, implementing, etc.) one or more scenes based on the one or more events (and/or lack of one or more events); and/or performing one or more output actions with the dog device, based on the one or more scenes (e.g., individual scenes; scene flows; etc.).

Additionally or alternatively, embodiments of the method 100 can include: accounting for the performance of one or more output actions (e.g., confirming the current status, such as position, of one or more components, such as mechanical actuators, at any given time and frequency such as in response to completion of one or more output actions; where the current status of one or more components can be used for event determination, scene determination, implementing instructions for performing one or more output actions, output action smoothing, and/or performing any suitable portion of embodiments of the method 100; etc.); generating one or more scene parameters; generating one or more event parameters; and/or any other suitable process.

Embodiments of the method 100 and/or the system 200 can function to determine and/or implement one or more actions for a dog device (e.g., a robotic dog device emulating live animal appearance and/or behavior; etc.) in the context of user inputs, such as for eliciting one or more user outcomes (e.g., emotional responses, medical outcomes, etc.).

Embodiments of the method 100 and/or the system 200 can be performed for characterizing (e.g., diagnosing; providing information relating to; etc.), for stimulating the production of endogenous oxytocin which is useful for treating, otherwise improving, and/or performed in any suitable manner for one or more conditions (e.g., for one or more users with one or more conditions; etc.) including one or more mental conditions (e.g., dementia, Alzheimer's, depression, anxiety, psychosis, bipolar disorder, ADD, ADHD, autism spectrum disorder, etc.) and/or other suitable conditions. In specific examples, any suitable portions of embodiments of the method 100 (e.g., causing the dog device to perform output actions, etc.) and/or any suitable portions of embodiments of the system 200 can be for facilitating improvement of one or more mental conditions through facilitating production of oxytocin in the user. In specific examples, embodiments can include using one or more dog devices to improve one or more states (e.g., symptoms, associated emotional states, etc.) of dementia (and/or other suitable medical conditions; etc.). In a specific example, embodiments can encourage users to develop an attachment to one or more dog devices based on realistic aesthetic (e.g., from external materials; mechanical design; etc.) and output actions (e.g., movement, audio; etc.), where the attachment can improve one or more states of dementia (and/or other suitable medical conditions; etc.), autism spectrum disorder, and/or other suitable mental conditions (e.g., described herein).

Embodiments can include and/or be used for a plurality of dog devices and/or other suitable dog devices. In specific examples, a first dog device can communicate with a second dog device (and/or any suitable number of dog devices), such as when the dog devices are within a threshold distance for Bluetooth communication and/or other suitable communication. In specific examples, scene types can include multi-device scene types, such as multi-dog device scene types associated with output actions of the dog devices interacting with each other (e.g., through mechanical output actions; through audio output actions; etc.). Interaction between dog devices can be associated with any suitable scene types (e.g., acknowledgement of another dog device; excited movement towards another dog device; looking at another dog device; howling at another dog device; etc.). Interactions between dog devices can encourage social interaction between users (e.g., users with dementia and/or other medical conditions; etc.), which can facilitate improvements in medical outcomes.

Embodiments can include collecting, analyzing, and/or otherwise using dog device usage data (e.g., describing how a user interacts with and/or otherwise uses one or more dog devices; etc.). Device usage data can include user input data, event-related data (e.g., amount, type, timing of, frequency, sequence of, and/or other aspects of events triggered by or not triggered by the user; etc.), scene-related data (e.g., amount, type, timing of, frequency, sequence of, and/or other aspects of scenes determined for and/or performed by the dog device for the user; user response to performed scenes, such as described by sensor input data collected by a dog device after performance of a scene; etc.), and/or other suitable data associated with a user. In specific examples, device usage data can be used to identify abnormal user behavior (e.g., based on abnormal trends and/or patterns detected in the device usage data, such as relative to the device usage data for one or more user populations; etc.), which can be used in facilitating diagnosis (e.g., facilitating diagnosis of a user as having a condition based on the user's device usage patterns resembling device usage patterns of a patient population with the condition; etc.) and/or treatment. In specific examples, device usage data (and/or associated insights from device usage data), can be transmitted and/or used by one or more care providers, such as for facilitating improved care for one or more users.

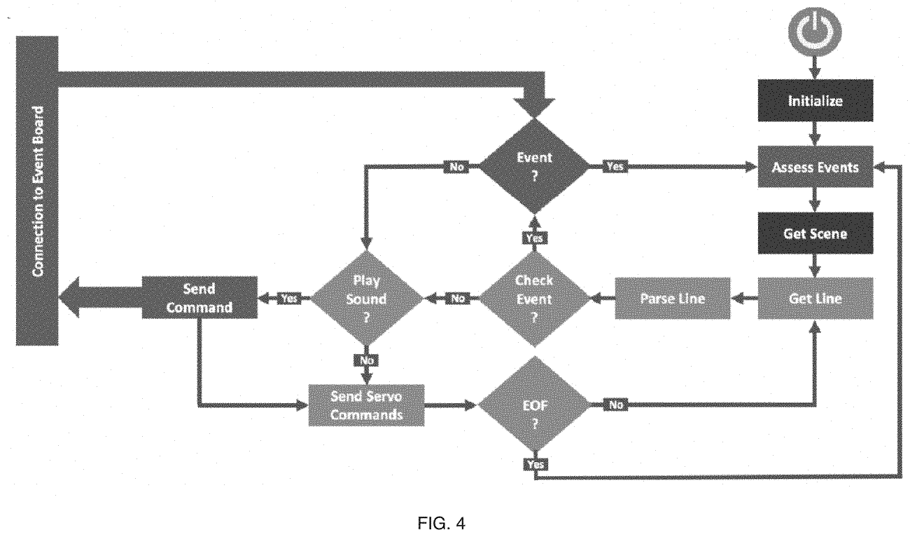

Embodiments of the method 100 and/or system 200 can include, determine, implement, and/or otherwise process one or more flows (e.g., logical flows indicating the sequence and/or type of action to perform in relation to operating the dog device; main flows associated with main operation of the dog device; event flows associated with events; scene flows associated with scenes; logic decision trees and/or any suitable type of logic framework; etc.). In a specific example, as shown in FIG. 4, a main flow can be implemented for detecting events, determining scenes based on events, and performing output actions based on scenes.

Additionally or alternatively, data described herein (e.g., input data, events, event-related data, scene types, scenes, output action-related data, flows, etc.) can be associated with any suitable temporal indicators (e.g., seconds, minutes, hours, days, weeks, time periods, time points, timestamps, etc.) including one or more: temporal indicators indicating when the data was collected, determined, transmitted, received, and/or otherwise processed; temporal indicators providing context to content described by the data; changes in temporal indicators (e.g., data over time; change in data; data patterns; data trends; data extrapolation and/or other prediction; etc.); and/or any other suitable indicators related to time.

Additionally or alternatively, parameters, metrics, inputs, outputs, and/or other suitable data can be associated with value types including any one or more of: classifications (e.g., event type; scene type; etc.), scores, binary values, confidence levels, identifiers, values along a spectrum, and/or any other suitable types of values. Any suitable types of data described herein can be used as inputs (e.g., for different models described herein; for portions of embodiments the method 100; etc.), generated as outputs (e.g., of models), and/or manipulated in any suitable manner for any suitable components associated with embodiments of the method 100 and/or system 200.

One or more instances and/or portions of embodiments of the method 100 and/or processes described herein can be performed asynchronously (e.g., sequentially), concurrently, in temporal relation to a trigger event (e.g., performance of a portion of the method 100), and/or in any other suitable order at any suitable time and frequency by and/or using one or more instances of embodiments of the system 200, components, and/or entities described herein.

Portions of embodiments of the method 100 and/or system 200 are preferably performed by a first party but can additionally or alternatively be performed by one or more third parties, users, and/or any suitable entities.

Any suitable disclosure herein associated with one or more dog devices can be additionally or alternatively analogously applied to devices of any suitable form (e.g., any suitable animal form, human form, any suitable robotic device, etc.).

In a specific example, the method 100 (e.g., for operating a dog device, etc.) can include receiving a first input, at a sensor of the dog device, from a user; determining an event based on the first input, the event comprising at least one of a touch event, a voice command recognition event, and a dog device position event; processing a scene based on the event, the scene including scene parameters indicating instructions for a first output action; and/or causing the dog device to perform the first output action based on the scene, wherein the first output action comprises at least one of a mechanical output action and an audio output action.

However, embodiments of the method 100 and/or system 200 can be configured in any suitable manner.

### 2.1 Receiving an Input.

Embodiments of the method 100 can include receiving one or more inputs at a dog device from one or more users and/or other suitable entities (e.g., additional dog devices; etc.), which can function to collect inputs for use in subsequent event, scene, and/or output action processing.

Inputs (e.g., input data; etc.) can include any one or more of touch inputs (e.g., at a region of the dog device; such as detected by touch sensors and/or buttons; etc.); audio inputs (e.g., voice commands; such as detected by audio sensors such as microphones, which can include omnidirectional and/or directional microphones; etc.); visual inputs (e.g., detected by optical sensors such as cameras; etc.); motion inputs (e.g., detected by motion sensors such as accelerometers and/or gyroscopes; etc.); and/or any suitable type of inputs.

Inputs can be received at one or more sensors of the dog device (e.g., where sensor input data is received; etc.), at a physical input receiving component (e.g., at a button of the dog device; etc.), at a base (e.g., a base connectable to the dog device; etc.), and/or at any suitable component (e.g., of the system 200; etc.). Sensor input data can include any one or more of: touch sensor data (e.g., capacitive sensor data; force sensor data; etc.), audio sensor data (e.g., microphone input data; omnidirectional microphone input data; directional microphone input data; etc.), optical sensor data (e.g., camera data; image sensor data; light sensor data; etc.), mechanical actuator sensor data (e.g., location sensor data (GPS receiver data; beacon data; indoor positioning system data; compass data; etc.), motion sensor data (e.g., accelerometer data, gyroscope data, magnetometer data, etc.), biometric sensor data (e.g., heart rate sensor data, fingerprint sensor data, facial recognition sensor data, bio-impedance sensor data, etc.), pressure sensor data, temperature sensor data, volatile compound sensor data, air quality sensor data, weight sensor data, humidity sensor data, depth sensor data, proximity sensor data (e.g., electromagnetic sensor data, capacitive sensor data, ultrasonic sensor data, light detection and ranging data, light amplification for detection and ranging data, line laser scanner data, laser detection and ranging data, etc.), virtual reality-related sensor data, augmented reality-related sensor data, and/or any other suitable type of sensor data.

Inputs are preferably received from one or more users (e.g., human users, etc.), but can additionally or alternatively be received from one or more animals (e.g., audio input and/or touch input from one or more animals; etc.), other devices (e.g., other dog devices, user devices, audio input and/or touch input from one or more devices, wireless and/or wired communication from other devices; etc.), and/or from any suitable entities. In a specific example, inputs can be received (e.g., at a wireless communication module of the dog device; etc.) via Bluetooth and/or any suitable wireless communication mechanism (e.g., WiFi, radiofrequency, Zigbee, Z-wave, etc.), such as for use in setting preferences (e.g., user preferences; emergency contacts, such as for communication when an emergency event is detected and/or an emergency scene is implemented; etc.) for the dog device, for controlling the dog device (e.g., to perform one or more output actions; etc.), for operating any suitable components (e.g., of embodiments of the system 200; etc.), and/or for any suitable purpose.

Inputs are preferably received for processing by one or more processing systems (e.g., a computer processing system of a dog device; control servers and/or event servers; etc.), but can be received for processing by any suitable component. In a specific example, inputs can be received for processing (e.g., a single computer processing system; multiple computer processing subsystems; etc.). In a specific example, inputs can be received for processing by one or more event boards (e.g., two event boards, etc.) of the dog device. Inputs can be received while a dog device is in a wait for event mode, and/or at any suitable time and frequency. In a specific example, the most recent input (e.g., out of a series of inputs, etc.) for an input-receiving component is stored (e.g., for use in event determination). In a specific example, after receiving an input (e.g., and storing the input for use in event determination), the input processing can be paused for a time limit (e.g., 5 seconds, any suitable amount of time; etc.), where any new inputs can be ignored during the time limit period. Pausing of input processing (e.g., pausing after receipt of a first input to process for event determination, etc.) can facilitate realistic output actions (e.g., realistic movement; realistic audio playback; etc.) by the dog device through smoothing out the performance of scenes and/or suitable output actions over time (e.g., by limiting the number of scenes performed over time; by allowing scenes to be fully performed; etc.). However, receiving inputs (and/or pausing processing of inputs) can be performed in any suitable manner.

However, inputs can be received in any suitable manner.

### 2.2 Determining an Event.

Embodiments of the method 100 can include determining one or more events (and/or a lack of one or more events), such as based on the one or more inputs (and/or a lack of one or more inputs), which can function to perform analyses upon collected data for facilitating subsequent scene determination and/or performance of output actions by a dog device.

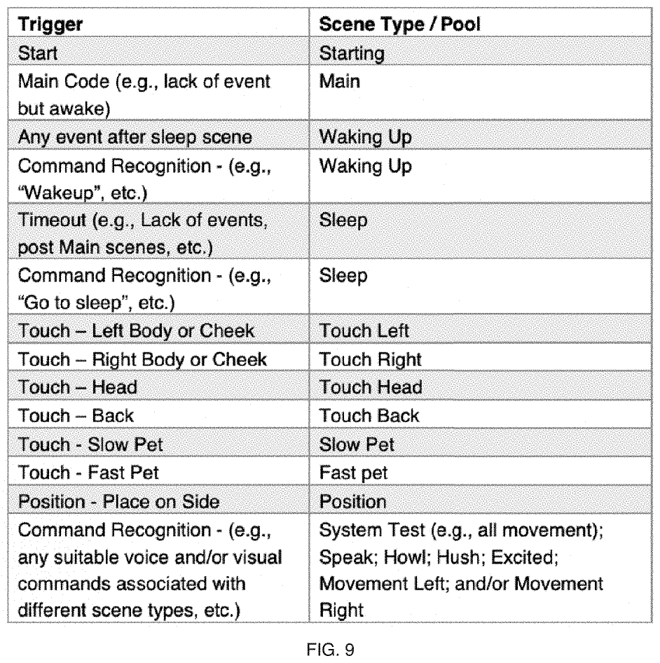

Events can be typified by one or more event types (e.g., in any suitable numerical relationship between number of events and number of event types; etc.) including any one or more of (e.g., as shown in FIG. 9, etc.): touch events; command recognition events; position events (e.g., associated with the dog device in a specified physical position, such as when a dog device has been placed on its side and/or other region; etc.); events associated with one or more flows (e.g., event flows; scene flows; main flows; such as a main code event associated with a main flow for the dog device; such as a start event associated with initialization of the dog device; etc.); events associated with one or more scenes (e.g., any event after a sleep scene; an event before, during, and/or after any suitable scene; etc.); lack of events; sensor input data-related events; non-sensor input data-related events; and/or any other suitable types of events.

In examples, touch events can include any one or more of: left body or cheek touch events (e.g., where touch inputs were received at the left body or cheek of the dog device; etc.); right body or cheek touch event; head touch events; back touch events; pet events (e.g., slow pet event; fast pet event; etc.); pressure-sensitive touch events (e.g., touch events differentiated by an amount of pressure associated with a touch event, such as indicated by a pressure touch sensor of a dog device; etc.); and/or any suitable type of touch events (e.g., where a given touch event can correspond to a given scene; etc.). In a specific example, determining one or more events can include determining a slow pet event or fast pet event (and/or any suitable pet speed event and/or type of petting event) based on the number of touch inputs received (e.g., at touch sensors, such as repeated touch inputs received at a same set of touch sensors; etc.) over a time period (e.g., indicating a rate of petting; etc.). In a specific example, the dog device includes a touch sensor, where the event includes a petting event including at least one of a slow petting event and a fast petting event, where determining the event includes determining the petting event based on a set of touch events received at the touch sensor over a time period, where processing the scene includes determining the scene based on the petting event. However, touch events can be processed in any suitable manner.

In examples, command recognition events (e.g., corresponding to recognition of voice commands from audio inputs; corresponding to visual commands indicated by optical sensor data; etc.) can include any one or more of (e.g., as shown in FIG. 9; etc.): wakeup commands (e.g., voice command including or associated with “wakeup” and/or suitable synonyms; corresponding to a waking up scene; etc.); sleep commands (e.g., voice command including or associated with “sleep” and/or suitable synonyms; corresponding to a sleep scene; etc.); system test commands (e.g., voice command including or associated with “system” and/or “test” and/or suitable synonyms; corresponding to a system test scene such as where one or more mechanical output actions, audio output actions, scene-associated output actions, are tested and/or evaluated; etc.); speak commands (e.g., voice command including or associated with “speak” and/or suitable synonyms; corresponding to a speak scene; etc.); sing commands (e.g., voice command including or associated with “sing” and/or suitable synonyms; corresponding to a howl scene; etc.); hush commands (e.g., voice command including or associated with “hush” and/or suitable synonyms; corresponding to a hush scene; etc.); play commands (e.g., voice command including or associated with “play” and/or suitable synonyms; corresponding to an excited scene; etc.); treat commands (e.g., voice command including or associated with “treat” and/or suitable synonyms; corresponding to an excited scene; etc.); movement commands (e.g., voice command including or associated with “look”, “move”, directionality such as “left”, “right”, “forward”, backward”, “up”, “down”, and/or suitable synonyms; corresponding to a movement scene such as a movement left scene or movement right scene; etc.); and/or any suitable command recognition events. In a specific example, where the dog device includes at least one audio sensor, where an event includes a voice command recognition event, where determining the event based on an input includes determining the voice command recognition event based on an audio input received at audio sensor(s) of the dog device, where processing the scene includes determining the scene based on the voice command recognition event, and where causing the dog to perform the output action includes causing the dog to simultaneously perform mechanical output action(s) and audio output action(s) based on the scene. However, command recognition events can be processed in any suitable manner.

Determining one or more events can include processing input data (e.g., mapping sensor input data to one or more events; determining one or more events based on input data; etc.). Processing a set of inputs (and/or any suitable portion of event determination); suitable portions of embodiments of the method 100; and/or suitable portions of embodiments of the system 200, can include, apply, employ, perform, use, be based on, and/or otherwise be associated with one or more processing operations including any one or more of: extracting features (e.g., extracting features from the input data, for use in determining events; etc.), performing pattern recognition on data (e.g., on input data for determining events; etc.), fusing data from multiple sources (e.g., from multiple sensors of the dog device and/or other components; from multiple users; etc.), combination of values (e.g., averaging values, etc.), compression, conversion (e.g., digital-to-analog conversion, analog-to-digital conversion), performing statistical estimation on data (e.g. ordinary least squares regression, non-negative least squares regression, principal components analysis, ridge regression, etc.), normalization, updating, ranking, weighting, validating, filtering (e.g., for baseline correction, data cropping, etc.), noise reduction, smoothing, filling (e.g., gap filling), aligning, model fitting, binning, windowing, clipping, transformations, mathematical operations (e.g., derivatives, moving averages, summing, subtracting, multiplying, dividing, etc.), data association, interpolating, extrapolating, clustering, sensor data processing techniques, image processing techniques (e.g., image filtering, image transformations, histograms, structural analysis, shape analysis, object tracking, motion analysis, feature detection, object detection, stitching, thresholding, image adjustments, etc.), other signal processing operations, other image processing operations, visualizing, and/or any other suitable processing operations.

Determining one or more events; suitable portions of embodiments of the method 100; and/or suitable portions of embodiments of the system 200 can include, apply, employ, perform, use, be based on, and/or otherwise be associated with artificial intelligence approaches (e.g., machine learning approaches, etc.) including any one or more of: supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.), reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminant analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and/or any suitable artificial intelligence approach. In examples, one or more artificial intelligence event models can be used for mapping (e.g., via a classification model; via a neural network model; etc.) input data (e.g., sensor input data; input data of different types; etc.) to one or more events (and/or event types; etc.). In a specific example, one or more event models and/or any other suitable models can be trained upon a user's inputs (e.g., to be able to recognize a user's voice, etc.) for user recognition, such as where scene determination based on events associated with the corresponding user's inputs can be personalized for that user (e.g., tailored to the corresponding user's preferences, needs, etc.).

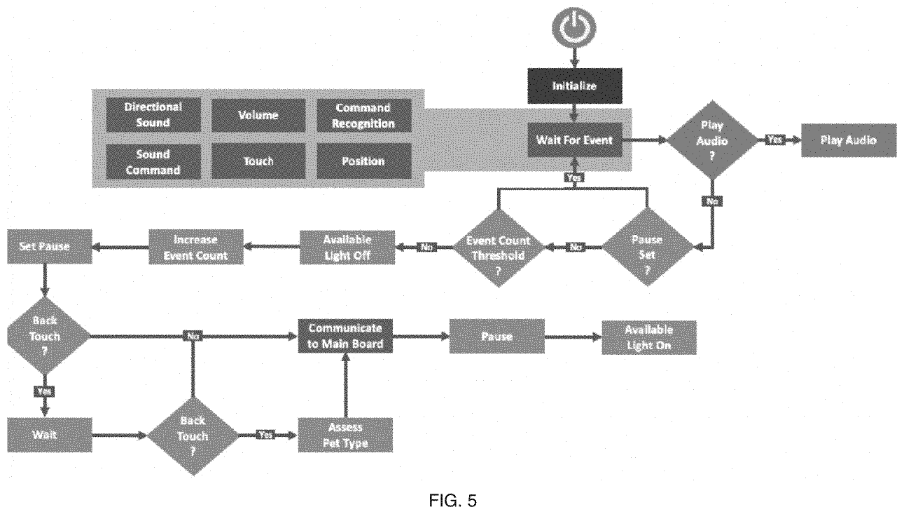

Determining one or more events can include (e.g., include implementation of; etc.) and/or be included as a portion of one or more event-related flows (e.g., event determination as one or more portions of one or more event-related flows; etc.), such as shown in a specific example in FIG. 5. In specific examples, the event-related flow in FIG. 5, and/or portions of the event-related flow can be used in differentiating between fast and slow pets (and/or between pets of any suitable speed and/or duration), such as where petting differentiation can trigger different suitable scene types and/or scenes. In a specific example, the method 100 can include: detecting a first touch input at least at one of a plurality of touch sensors (e.g., at least two sensors); and waiting a suitable time period (e.g., a threshold time period of any suitable amount of time; etc.) for a second touch input (e.g., a stroke, etc.) at least at one of the plurality of touch sensors (e.g., where corresponding events and associated scenes are not processed until after the suitable time period has elapsed; etc.). In a specific example, if no second touch input is detected over the time period, a touch event (e.g., instead of a petting event; etc.) is determined. In a specific example, if a second touch input is detected, then a petting event is determined, where the petting event can be a fast petting event (e.g., if the second touch input is detected soon after the first touch input, such as within a fast petting time threshold; etc.) or a slow petting event (e.g., if the second touch input is detected a longer time after the first touch input, such as after the fast petting time threshold but within the slow petting time threshold; etc.), and/or can be any suitable type of petting event (e.g., associated with any suitable speed; etc.). However, petting events can be determined in any suitable manner.

Additionally or alternatively, detection and/or analysis of any suitable events (and/or input data) can be monitored in any suitable sequence at any suitable time and frequency. However, event-related flows can be configured in any suitable manner.

Additionally or alternatively, embodiments of the method 100 can include determining a lack of one or more events (e.g., where determining a lack of input-triggered events can correspond to determining a timeout event; etc.). Determining a lack of one or more events can include determining a lack of one or more inputs (e.g., a lack of a set of inputs of a type triggering detection of an event; etc.). In examples, determining a lack of one or more events can be in response to a lack of one or more inputs over a threshold period of time (e.g., any suitable period of time; etc.). In examples, determining a lack of one or more events (e.g., determining a timeout event; etc.) can trigger one or more scenes (e.g., one or more scenes from a “sleep” scene type; etc.), but any suitable scenes and/or scene types can be determined based on a lack of one or more events (e.g., a lack of any events; a lack of specific event types; triggering a “Main Scene” in response to a lack of events while the dog device is in a non-sleep, awake mode; etc.). In a specific example, the method 100 can include monitoring for one or more inputs at a set of sensors of the dog device; determining a lack of one or more inputs after a predetermined time period threshold; and determining a sleep scene (and/or other suitable scene) based on the lack of the one or more inputs.

However, determining a lack of one or more events can be performed in any suitable manner.

Determining one or more events can be performed continuously; at specified time intervals; in response to one or more triggers (e.g., in response to receiving a threshold amount and/or type of inputs; in response to receiving any inputs; in response to receiving sensor input data; in response to initialization of the dog device; in response to completion of performance of one or more output actions, such as corresponding to one or more scenes; etc.); before, after, and/or during one or more events, scenes, flows (e.g., main flows, event flows, scene flows; etc.) and/or at any suitable time and frequency.

Determining one or more events is preferably performed by an event board (e.g., of a computing system of a dog device; etc.), but can additionally or alternatively be determined by any suitable component.

However, determining one or more events can be performed in any suitable manner.

### 2.3 Processing a Scene.

Embodiments of the method 100 can include processing one or more scenes, which can function to determine, implement, sequence, and/or otherwise process one or more scenes, such as for guiding performance of one or more output actions by one or more dog devices.

Scenes (and/or scene types; etc.) preferably include one or more scene parameters (e.g., stored in a scene file for a scene; etc.) indicating instructions for one or more output actions (e.g., mechanical output actions; audio output actions; etc.). In a specific example, scene parameters can include one or more servos (and/or suitable mechanical actuator) parameters (e.g., indicated by numerical values; code; etc.) for operating position, speed, timing (e.g., when to perform the mechanical output actions; etc.), and/or other suitable parameters for mechanical output components (e.g., for instructing one or more mechanical output actions by the dog device; etc.). In a specific example, scene parameters can include one or more audio (e.g., emitted by a speaker of the dog device; etc.) parameters (e.g., indicated in a different or same file for mechanical actuator parameters, such as indicated by an identifier identifying one or more audio files to play for a scene; etc.) for operating the type of audio output played (e.g., the audio file to play), volume, pitch, tone, timing (e.g., when to play the audio; stopping audio output during transition to a new scene; etc.), directionality, speaker selection (e.g., from a set of speakers of a dog device; etc.), speed, and/or other suitable parameters for audio output actions.

In specific examples, scene types can be associated with sets of scene parameters (e.g., specified ranges for mechanical output parameters and/or audio output parameters, where such ranges can be associated with a dog device output action performance representative of the scene type; where such ranges can be selected from for generating one or more scenes for the corresponding scene type; etc.)

Scenes and/or scene types can be associated with any suitable indicators and/or identifiers (e.g., prefixes such as letters; names numbers; combinations of characters; graphical identifiers; audio identifiers; verbal identifiers, etc.). In a specific example, scene types can be associated with one or more prefixes (e.g., one or more letters; where a given scene type is associated with a given prefix; etc.), where such prefixes can correspond to one or more scene types and accordingly corresponding to one or more scenes (e.g., where a scene type and prefix can be associated with a plurality of scenes; etc.).

As shown in FIG. 9, scene types can include any one or more of: starting scene types; main scene types; waking up scene types; sleep scene types; touch scene types (e.g., touch scene types for any suitable region of the dog device; etc.); petting scene types (e.g., slow pet scene types; fast pet scene types; petting scene types for any suitable petting speed and type; etc.); position scene types (e.g., for any suitable dog device position; etc.); system test scene types (e.g., for testing and/or evaluating one or more output actions and/or other suitable components of the dog device; etc.); speak scene types; howl scene types; hush scene types; excited scene types; movement scene types (e.g., movement scene types for any suitable directionality, distance, and/or type of movement; etc.); multi-device scene types (e.g., for scenes between a plurality of dog devices and/or other suitable device; etc.); and/or any suitable types of scenes.

Scene types can include any number of different scenes (e.g., for enabling random and/or guided selection of a scene for a given scene type; for facilitating a variety of output actions for a given scene; for improving user perception of the dog device as a natural entity; etc.). In an example, a scene type can include any number of different scenes corresponding to different sets of mechanical, audio, and/or other suitable outputs for performing a given scene type; etc.). Processing one or more scenes can include determining a scene type based on an event (e.g., a determined event; etc.); determining a scene based on the scene type; and/or performing one or more output actions at the dog device based on the scene. Determining one or more scenes for one or more scene types can include randomly selecting one or more scenes for the one or more scene types. In examples, scenes can be selected based on, indicated by, and/or identified by one or more identifiers (e.g., a count; letters; names; numbers; combinations of characters; graphical identifiers; audio identifiers; verbal identifiers, etc.). Scene parameters, files, associated indicators and/or identifiers, and/or any suitable scene-related data can be stored at one or more storage components of the dog device and/or any suitable devices.

Determining one or more scene types (and/or scenes) is preferably based on one or more events (e.g., events triggering one or more scene types and/or scenes; etc.). In specific examples, specific event triggers can map to specific scene types, as shown in FIG. 9. Additionally or alternatively, any number and/or type of event triggers can map to any number and/or type of scene types, where any suitable numerical relationship (e.g., 1:many, many:1, 1:1, etc.) can be used for associations between event triggers and scene types. Scene types (e.g., corresponding scenes; etc.) can be selected based on any suitable number and/or type of events (e.g., event triggers; etc.) detected at any suitable time and frequency. In a specific example, any suitable scene type can be associated with event monitoring (e.g., for determining one or more events; etc.), such as at one or more time periods during performance of the scene. In a specific example, any suitable scene type can be triggered in response to determining a lack of events. In a specific example, repetition of scene performance can trigger one or more scene types, but any suitable sequence of scene performance can trigger any suitable scene types. Main scenes and/or any suitable scene types can be associated with timeout events (e.g., lack of events over a period of time; lack of events over a threshold number of performances of one or more scenes such as main scenes; etc.), where such timeout events can trigger any suitable scene type.

Additionally or alternatively, determining scene types (and/or scenes) can be based on any suitable data (e.g., input data not used for determining events; user preferences; dog device settings; dog device output action capability, such as where different sets of scenes can be selectable based on the version of a dog device; etc.).

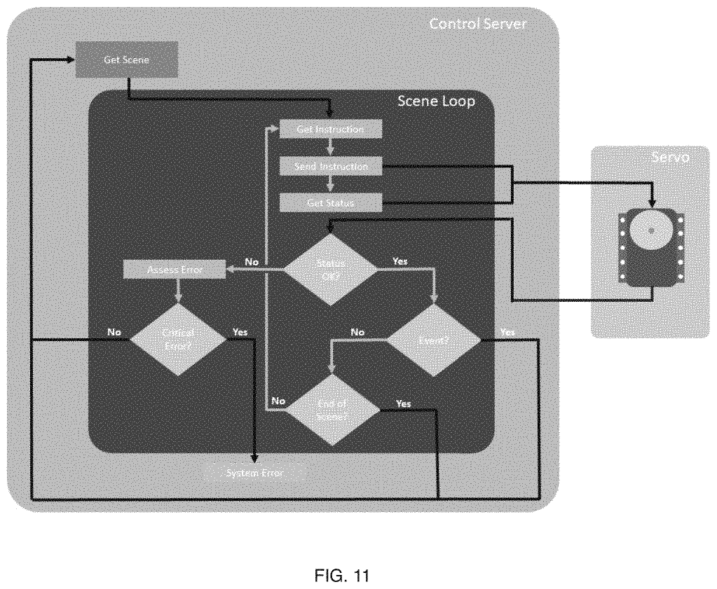

Processing a scene and/or other suitable portions of embodiments of the method 100 and/or system 200 can be associated with mechanical actuator sensors of the mechanical actuators of the dog device (and/or other suitable components). Mechanical actuator sensors can function to facilitate the safety of users, the dog device, and/or any other suitable entities. Mechanical actuator sensors can be used for determining position data (e.g., positions of the mechanical actuators; positions of components of the dog device; etc.), temperature data (e.g., temperatures of the mechanical actuators; temperatures of components of the dog device; etc.), strain data (e.g., strain associated with the mechanical actuators and/or components of the dog device; etc.), and/or other suitable types of data. Mechanical actuator sensors can collect data at any suitable time and frequency. In a specific example, mechanical actuator sensors can collect data during performance of instructions by the mechanical actuators (e.g., to move to a particular position, at a particular time, a at particular speed, etc.). Mechanical actuator sensor data can be used to determine one or more statuses associated with performance of output actions, where statuses can include a normal status, different types of errors (e.g., overheating, high torque/current, high strain; etc.), and/or other suitable statuses. In a specific example, as shown in FIG. 11, the method 100 can include after providing instructions (e.g., indicated by scene parameters, etc.) to one or more mechanical actuators of the dog device, determining a status based on mechanical actuator data collected by corresponding mechanical actuator sensors, associated with performance of the instructions. In a specific example, if the status includes a normal status, additional information can be retrieved, such as one or more of: occurrence of an event (e.g., and if so, the associated scene; etc.), a subsequent scene (e.g., if there was the end of an initial scene; etc.), subsequent instructions in a loop and/or flow, and/or other suitable information. In a specific example, if the status includes an error status, actions can be performed to address the errors, where actions can include one or more of: powering off the mechanical actuators in response to overheating; in response to high torque or current (e.g., indicating that the mechanical actuator(s) are under stress; etc.), analyzing current direction of movement and re-directing the movement of the mechanical actuator(s) (e.g., by retrieving an applicable scene; etc.), or pausing movement (e.g., in response to errors, and/or mechanical actuator sensor data satisfying a threshold condition); in response to critical errors, exiting loops and/or flows and entering a larger system error flow.

In an example, the dog device includes a set of mechanical actuators including a set of mechanical actuator sensors, where the method 100 can include causing the dog device to perform the first output action with the mechanical actuators based on the scene; receiving mechanical actuator sensor data during the performance of the first output action by the dog device; determining a status of the performance of the first output action by the dog device based on the mechanical actuator sensor data; and causing the dog device to perform a second output action (e.g., a modified mechanical output action to prevent harm to the user and/or dog device; a modified audio output action; etc.) based on the status of the performance of the first output action by the dog device. In an example, determining a status of the performance of the first output action by the dog device includes determining a status of the performance of the first output action by the dog device based on the mechanical actuator sensor data during performance of the scene by the dog device, and where causing the dog device to perform the second output action includes causing the dog device to perform a modified version of the first output action for completion of the scene. In an example, determining the status of the performance of the first output action and causing the dog device to perform the second output action are for facilitating improvement of safety of the user and the dog device. In an example, the method 100 can additionally or alternatively include determining strain and temperature associated with the set of mechanical actuator sensors based on the mechanical actuator sensor data, where the strain and temperature are associated with the performance of the first output action, and where determining the status of the performance of the first output action by the dog device includes determining the status of the performance of the first output action based on the strain and temperature associated with the set of mechanical actuator sensors. However, utilizing the mechanical actuator sensors can be performed in any suitable manner.

Determining one or more scenes can be based on a lack of one or more events (e.g., over a time period, such as a predetermined and/or automatically determined time period; etc.), such as shown in FIG. 9 (e.g., where lack of events during an awake mode can trigger a main scene type; where a timeout event can trigger a sleep scene type; etc.).

Additionally or alternatively, processing one or more scenes (e.g., mapping one or more events and/or input data to one or more scene types and/or scenes; etc.); suitable portions of embodiments of the method 100; and/or suitable portions of the system 200 can include, apply, employ, perform, use, be based on, and/or otherwise be associated with one or more processing operations including any one or more of: extracting features, performing pattern recognition on data, fusing data from multiple sources, combination of values (e.g., averaging values, etc.), compression, conversion (e.g., digital-to-analog conversion, analog-to-digital conversion), performing statistical estimation on data (e.g. ordinary least squares regression, non-negative least squares regression, principal components analysis, ridge regression, etc.), normalization, updating, ranking, weighting, validating, filtering (e.g., for baseline correction, data cropping, etc.), noise reduction, smoothing, filling (e.g., gap filling), aligning, model fitting, binning, windowing, clipping, transformations, mathematical operations (e.g., derivatives, moving averages, summing, subtracting, multiplying, dividing, etc.), data association, interpolating, extrapolating, clustering, sensor data processing techniques, image processing techniques (e.g., image filtering, image transformations, histograms, structural analysis, shape analysis, object tracking, motion analysis, feature detection, object detection, stitching, thresholding, image adjustments, etc.), other signal processing operations, other image processing operations, visualizing, and/or any other suitable processing operations.

Determining one or more events; suitable portions of embodiments of the method 100; and/or suitable portions of embodiments of the system 200 can include, apply, employ, perform, use, be based on, and/or otherwise be associated with artificial intelligence approaches (e.g., machine learning approaches, etc.) including any one or more of: supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.), reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminant analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and/or any suitable artificial intelligence approach. In a specific example, scene models (e.g., classification models; decision tree models; neural network models; etc.) can be applied for mapping event-related features and/or event determinations (and/or other suitable data) to one or more scenes and/or scene types. In a specific example, scene models (e.g., classification models; decision tree models; neural network models; etc.) can be applied for mapping input data (e.g., sensor input data; etc.) (and/or other suitable data) directly to one or more scenes and/or scene types.

Processing one or more scenes is preferably performed in relation to (e.g., in response to; after; etc.) determining one or more events (e.g., one or more events mappable to one or more scene types and/or scenes; etc.) and/or lack of one or more events, but can additionally or alternatively be performed at any suitable time and frequency (e.g., in relation to and/or as part of any suitable scene flows, main flows, event-related flows; etc.).

Determining one or more scenes can include sequencing one or more scenes (e.g., where the dog device can perform one or more output actions in an order corresponding to the sequencing of the one or more scenes; etc.). In a specific example, scene processing can be performed in relation to the timing of event determination (e.g., ignoring inputs for a period of time, such as 5 seconds, in response to determination of an event and/or scene, such as where collection of input data can be restarted after the period of time; etc.). In a specific example, scene sequencing can be randomized (e.g., across different scene types; within a given scene type; randomization of scenes within a scene implementation queue; etc.). In a specific example, scene processing can be based on event count (e.g., foregoing performance of a scene type in response to consecutive detection of events mapping to that scene type beyond a threshold number; etc.) and/or any suitable event-related data. In a specific example, scenes can be sequenced based on detected order of events (e.g., determining, in order, a first, second, and third event; and determining a sequence of a first, second, and third scene respectively corresponding the first, second, and third event; etc.) and/or input data. In a specific example, scene sequencing can be based on a ranking of scenes (e.g., where a first scene can be prioritized for implementation over a second scene that was determined prior to the first scene; ranked based on input data; etc.). In a specific example, scene sequencing can be personalized to one or more users (e.g., prioritizing one or more scenes based on a user preference of such scenes; etc.). However, sequencing one or more scenes can be performed in any suitable manner.

Processing one or more scenes can include processing one or more scene flows (e.g., applying scene logic for determination of one or more scenes and/or associated sequences; determining one or more scene flows to implement; etc.).

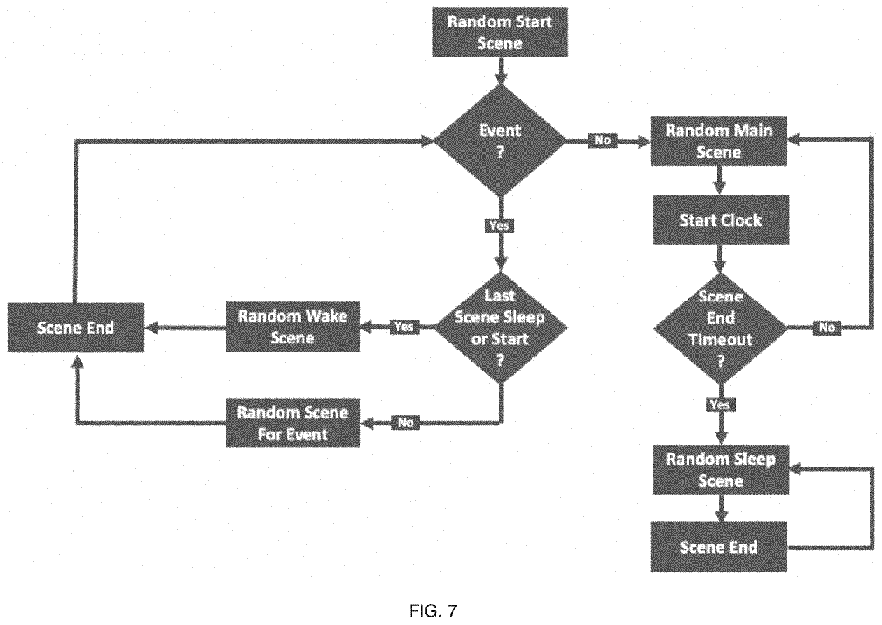

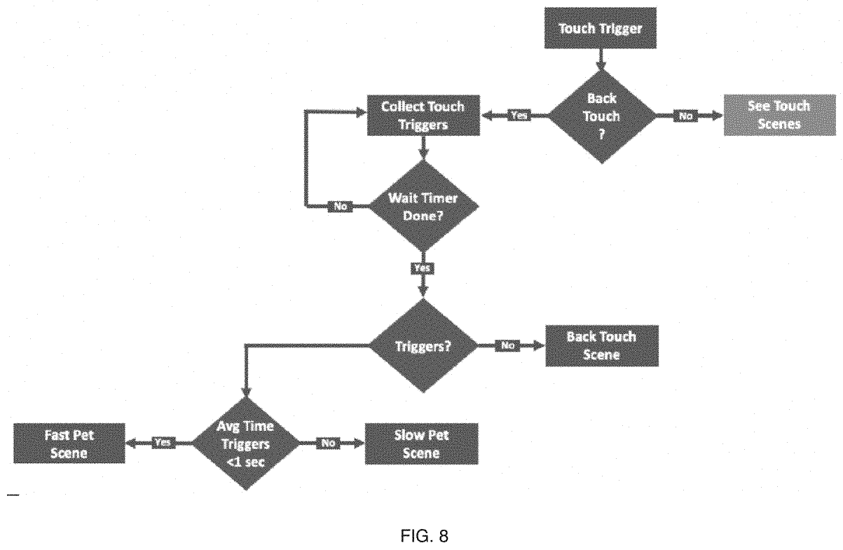

As shown in FIG. 7-8, scene flows preferably include a set of scenes to be performed according to scene logic (e.g., sequences for the scenes; triggers for the scenes; etc.), but can additionally or alternatively include any other suitable parameters and/or components.

Scene flows can include one or more scene flows for event assessment (e.g., to be performed during a time period associated with event evaluation, etc.), such as shown in a specific example in FIG. 7.

Scene flows can include one or more petting scene flows (e.g., to be performed according to petting scene logic), such as shown in a specific example in FIG. 8.

Processing one or more scenes can include implementing one or scenes (e.g., sending commands for audio output actions, such as sending instructions to a computer processing system, such as including event board and/or other processing system, for playing one or more audio outputs; sending commands for mechanical output actions, such as sending servo commands, via the computer processing system, such as including an action board and/or other processing system, for controlling one or more servo devices of the dog device; etc.), such as shown in FIG. 4.

Processing one or more scenes is preferably performed at a computational processing system of the dog device. Additionally or alternatively, processing one or more scenes can be performed at one or more action boards (e.g., of a computing system of the dog device; etc.), but can additionally or alternatively be performed by any suitable components.

However, processing one or more scenes can be performed in any suitable manner.

### 2.4 Performing an Output Action.

Embodiments of the method 100 can include performing one or more output actions with one or more dog devices, which can function to perform one or more scenes and/or other suitable actions (e.g., for eliciting one or more user outcomes, such as emotional responses and/or medical outcomes; etc.). In a specific example, one or more output actions can simulate real dog aesthetic and actions (e.g., movement, sound, etc.), such as for facilitating an emotional attachment from a user to the dog device, which can thereby improve a state of dementia and/or other suitable conditions.

Types of output actions can include any one or more of: mechanical output actions (e.g., performed using one or more mechanical output components, such as servos mechanical actuators; etc.), audio output actions (e.g., performed using one or more audio output components, such as one or more speakers; etc.), graphical output actions (e.g., performed using one or more graphic displays; etc.), communication output actions (e.g., communication to one or more user devices, such as notifications, etc.), and/or any suitable output actions.

Performing one or more output actions is preferably based on one or more scenes (e.g., for implementation of the one or more scenes). In specific examples, scenes can include one or more scene parameters (e.g., stored in one or more corresponding scene files; etc.) for operating one or more mechanical output components (e.g., mechanical actuators; servos; etc.); one or more audio output components (e.g., speakers; etc.); and/or other suitable output components used in performing one or more output actions (e.g., where the scene parameters can include and/or be used for generating instructions for the one or more output components; etc.). Additionally or alternatively, performing one or more output actions can be based on any suitable data (e.g., output actions as a component of main flows, event flows, scene flows, etc.).

In variations, performing one or more output actions can include smoothing (and/or otherwise modifying) one or more output actions, such as based on modifying speed, position, and/or suitable parameters (e.g., scene parameters; etc.). In specific examples, smoothing can include performing one or more transition output actions for transitioning into, out of, and/or between one or more scenes (and/or suitable output actions; etc.). Different scenes, scene type, and/or output actions can be associated with different types of smoothing (e.g., linear soothing; acceleration, deceleration, and/or different speeds for different portions of scenes, for different scenes, for different scene types; etc.). However, smoothing and/or otherwise modifying one or more scenes, scene types, and/or output actions can be performed in any suitable manner.

Performing one or more output actions is preferably performed at a dog device (e.g., where the dog device performs the mechanical movement and/or playback of audio; etc.), but can additionally or alternatively be performed at any suitable component (e.g., where instructions to play audio is communicated to a user device, for playback at the user device; etc.). Processing of instructions for mechanical output actions is preferably performed at an action board (e.g., of a computing system of a dog device; etc.), but can additionally or alternatively be performed at any suitable component. Processing of instructions for audio output actions is preferably performed at an event board (e.g., of a computing system of a dog device; etc.), but can additionally or alternatively be performed at any suitable component. Additionally or alternatively, any suitable output actions can be processed and/or performed at any suitable components.

However, performing one or more output actions can be performed in any suitable manner.

However, embodiments of the method 100 can be performed in any suitable manner.

### 3. System.

Embodiments of the system 200 can include one or more: dog devices 205, dog device attachments 206 (e.g., a base and/or other component physically and/or wirelessly connectable to one or more dog devices 205; a base attachment upon which a dog device 205 can be positioned; etc.), remote computing systems (e.g., for storing and/or processing data; for communicating with one or more dog devices 205, dog device attachments 206, and/or other suitable components; etc.), and/or other suitable components.

Embodiments of the system 200 and/or portions of embodiments of the system 200 can entirely or partially be executed by, hosted on, communicate with, and/or otherwise include one or more: remote computing systems (e.g., one or more servers, at least one networked computing system, stateless, stateful; etc.), local computing systems, user devices (e.g., mobile phone device, other mobile device, personal computing device, tablet, wearable, head-mounted wearable computing device, wrist-mounted wearable computing device, etc.), databases, application programming interfaces (APIs) (e.g., for accessing data described herein, etc.) and/or any suitable components. Communication by and/or between any components of the system and/or other suitable components can include wireless communication (e.g., WiFi, Bluetooth, radiofrequency, Zigbee, Z-wave, etc.), wired communication, and/or any other suitable types of communication.

Components of embodiments of the system 200 can be physically and/or logically integrated in any manner (e.g., with any suitable distributions of functionality across the components, such as in relation to distributions of functionality across event boards, action boards, single computational processing systems, control server(s), event server(s) and/or other suitable components; across portions of embodiments of the method 100; etc.).

Dog devices 205, dog device attachments 206, and/or other suitable components can include any number of sensors 210, output action components (e.g., components for performing one or more output actions; mechanical actuators 230 such as servos; mechanical actuators 230 providing any suitable degrees of freedom of movement; speakers 240; etc.), computing systems, storage components, and/or other suitable components.

In variations, components (e.g., sensors 210, output action components, computing systems, storage components, etc.) of embodiments of the system 200 can be positioned at (e.g., mounted at, integrated with, located proximal, etc.) any suitable location (e.g., any suitable region of the dog device 205; of the dog device attachment 206; etc.) and/or oriented in any suitable manner. In specific examples, mechanical output components can be positioned and/or oriented to emulate live dog anatomy and/or bone structure (e.g., positioning and orienting servos at regions where live dogs bend and move; etc.). In specific examples, a dog device 205 can be constructed with materials (e.g., external materials, etc.), design (e.g., material design; mechanical design; etc.), mechanical output components (e.g., operated based on performance of portions of embodiments of the method 100, etc.), and/or suitable components with suitable positioning and/or orientation (e.g., emulating a real dog neck region in relation to aesthetic and movement; etc.) for facilitating realistic looking and acting of the dog device 205, which can encourage a user to form an attachment (e.g., emotional attachment) with the dog device 205 and thereby improve a state of dementia (and/or other suitable conditions).

Additionally or alternatively, components of the system 200 can be integrated with any suitable existing components (e.g., existing charging devices; existing user devices; etc.).

Components of the system can be manufactured using any one or more of: molding (e.g., injection molding, etc.), microlithography, doping, thin films, etching, bonding, polishing, patterning, deposition, microforming, treatments, drilling, plating, routing, CNC machining & casting, stereolithography, Digital Light Synthesis, additive manufacturing technologies, Fused Deposition Modeling (FDM), suitable prototyping approaches, and/or any other suitable manufacturing techniques. Components of the system can be constructed with any suitable materials, including recyclable materials, plastics, composite materials, metals (e.g., steel, alloys, copper, etc.), glass, wood, rubber, ceramic, flexible materials (e.g., for the eyebrows of the head region of the dog device 205; for fur of the dog device 205; etc.), rigid materials, and/or any other suitable materials.

A dog device 205 can include a neck region, which can function to enable mechanical movement associated with a neck of a dog device 205 (e.g., for performance of one or more output actions; etc.). In specific examples, the neck region can emulate a real dog neck region with specific materials (e.g., external materials, etc.), design (e.g., material design; mechanical design; etc.), mechanical output components (e.g., operated based on performance of portions of embodiments of the method 100, etc.), and/or suitable components with suitable positioning and/or orientation. The neck region can include any suitable number of mechanical output components positioned at the neck region and oriented in any suitable manner (e.g., seven servos positioned at the neck region; any suitable number of servos at the neck region; providing any suitable degrees of freedom of movement, such as at least freedom of movement in the x, y, and z axes; etc.). In specific examples, the neck region can include mechanical output components for providing pivot and/or tilt capability at any suitable joints (e.g. top joint of the neck region; bottom region of the neck region; etc.). However, the neck region can be configured in any suitable manner.

A dog device 205 can include a head region, which can function to enable mechanical movement associated with a head of a dog device 205 (e.g., for performance of one or more output actions; etc.). The head region can include any suitable number of mechanical output components positioned at the head region and oriented in any suitable manner (e.g., four servos positioned at the head region, such as for controlling ears and eyebrows of the head region; two servos at the head region, one for each of controlling the ears and the eyebrows; any suitable number of servos at the head region; providing any suitable degrees of freedom of movement; etc.). In a specific example, material of the eyebrow (and/or suitable component of the dog device 205; etc.) can be physically connected to one or more mechanical output components (e.g., servos; etc.), such as for performing one or more output actions associated with moving the material (e.g., lifting the eyebrows to open the eye of the dog device 205; etc.). In a specific example, a mechanical output component can be physically connected to a mouth of the dog device 205 (e.g., for opening and closing the mouth; etc.). In a specific example, the mouth can include one or more springs and/or force softening components (e.g., positioned at the bottom of the mouth; etc.), such as to prevent full closure of the mouth onto a user body region. However, the head region can be configured in any suitable manner.

A dog device 205 can include a body region, which can function to enable mechanical movement associated with a body of a dog device 205 (e.g., for performance of one or more output actions, such as for emulating breathing, walking, turning; etc.). The body region can include any suitable number of mechanical output components positioned at the body region and oriented in any suitable manner (e.g., two servos positioned at the body region; any suitable number of servos; providing any suitable degrees of freedom of movement; etc.). However, the body region can be configured in any suitable manner.

A dog device 205 can include a tail region, which can function to enable mechanical movement associated with a tail of a dog device 205 (e.g., for performance of one or more output actions, such as for emulating tail wagging; etc.). The tail region can include any suitable number of mechanical output components positioned at the tail region and oriented in any suitable manner (e.g., two servos positioned at the tail region for lifting the tail and wagging the tail to the left and right, respectively; any suitable number of servos; providing any suitable degrees of freedom of movement; etc.). The tail region can include any suitable mechanical components for providing one or more hinges (e.g., for creative a pivot point for emulating natural movement of a tail; etc.). However, the tail region can be configured in any suitable manner.

A dog device 205, dog device attachment 206, and/or suitable components of embodiments of the system 200 can include any number and/or type of sensors 210 positioned at any suitable location and/or oriented in any suitable manner. Sensors 210 can include any one or more of: touch sensors 211 (e.g., capacitive sensors; force sensors; etc.), audio sensors 212 (e.g., microphones; omnidirectional microphones; directional microphones; microphones at the dog device 205, such as near the head region of the dog device 205; microphones at a dog device attachment 206; etc.), optical sensors (e.g., cameras; image sensors; light sensors 213, such as where light sensor data can be used to modify performance of one or more output actions, such as decreasing the volume of audio output actions in response to detecting nighttime based on the light sensor data; etc.), location sensors (GPS receivers; beacons; indoor positioning systems; compasses; etc.), motion sensors (e.g., accelerometers, gyroscopes, magnetometers; for detecting a tip over event when the dog device 205 tips over, which can be used for triggering any suitable scene types such as a sleep scene type; etc.), biometric sensors 215 (e.g., heart rate sensors, fingerprint sensors, facial recognition sensors, bio-impedance sensors, etc.), pressure sensors, temperature sensors, volatile compound sensors, air quality sensors, weight sensors, humidity sensors, depth sensors, proximity sensors (e.g., electromagnetic sensors, capacitive sensors, ultrasonic sensors, light detection and ranging, light amplification for detection and ranging, line laser scanner, laser detection and ranging, etc.), virtual reality-related sensors, augmented reality-related sensors, and/or or any other suitable type of sensors 210. In specific examples, sensors 210 of a dog device 205 can include a set of touch sensors 211 (e.g., two touch sensors at the head region, including a sensor on each cheek; four touch sensors across the back region; a touch sensor on each side of the body region; a touch sensor at the tail region; touch sensors at the ears, paws, face, nose, muzzle; and/or any suitable touch sensors 211 at any suitable location). In a specific example, touch sensors 211 can include capacitive touch sensors. Additionally or alternatively, touch sensors 211 can include copper foil sensors and/or any suitable type of touch sensors 211. In a specific example, the set of sensors 210 of the dog device 205 includes: at least one touch sensor, at least one audio sensor 212, at least one light sensor 213, and at least one mechanical actuator sensor 214.

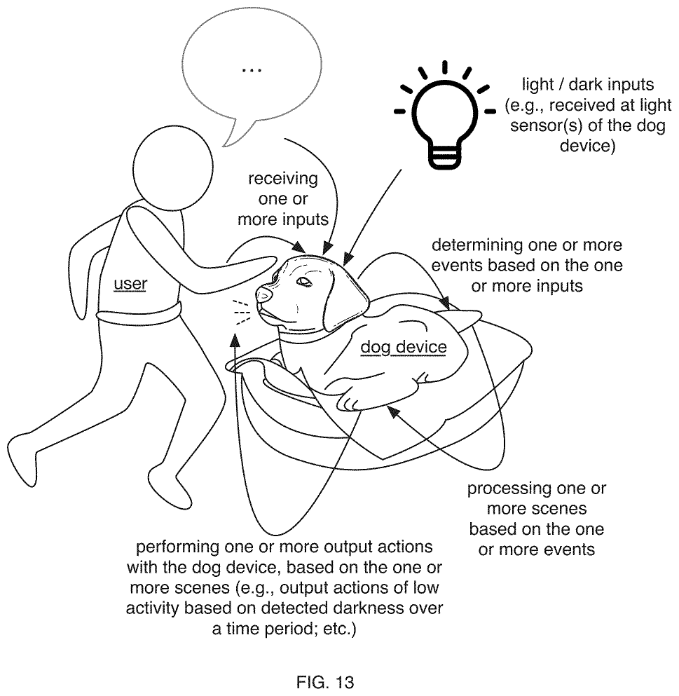

In examples, as shown in FIG. 13, the system 200 (e.g., dog device 205, a dog device attachment 206 206, etc.) and/or method 100 can include and/or utilize one or more light sensors 213 for detecting light, darkness, day, night, etc., such as for event determination and/or scene determination. In an example, the sensor input data includes light sensor data (e.g., indicating darkness, etc.), where processing a scene includes determining a scene associated with a low activity level for the output action(s) by the dog device 205, based on the light sensor data. In a specific example, scenes associated with low activity (e.g., decreased speaker volume, decreased movement from mechanical output actions, etc.) can be determined based on light sensor data indicating darkness (e.g., satisfying a threshold level of darkness) over a time period (e.g., satisfying a threshold time period). However, light sensors 213 can be utilized in any suitable manner and in relation to any suitable portions of embodiments of the method 100 and/or system 200.

In examples, the system 200 where the set of sensors 210 of the dog device 205 includes: at least one touch sensor 211 and at least one audio sensor 213; at least one mechanical actuator sensor 214 for receiving mechanical actuator sensor data, where the processing system 220 is operable to determine updated scene parameters based on the mechanical actuator sensor data; at least one light sensor 213 for receiving light sensor data, where the processing system 220 is operable to determine the scene based on the light sensor data.

In examples, the system 200 (e.g., the dog device 205, a dog device attachment 206, etc.) can include one or more biometric sensors 215, which can function to facilitate user monitoring (e.g., patient health monitoring), such as remote user monitoring, and/or medical characterization. In examples, the system 200 can include at least one biometric sensor 215 (e.g., at the dog device 205, etc.) for collecting medical-related data from the user for characterizing at least one of: heart arrhythmia, heart rate variation, blood pressure, respirations, temperature, blood oxygen levels, blood glucose levels, sepsis detection, seizures, stroke, fall detection, and sleep monitoring. However, biometric sensors 215 can be utilized in any suitable manner and in relation to any suitable portions of embodiments of the method 100 and/or system 200.

Sensors 210 can be connected to any suitable components of the computing system (e.g., a board at the head region; a board at the body region; etc.) and/or components of embodiments of the system 200. However, sensors 210 can be configured in any suitable manner.

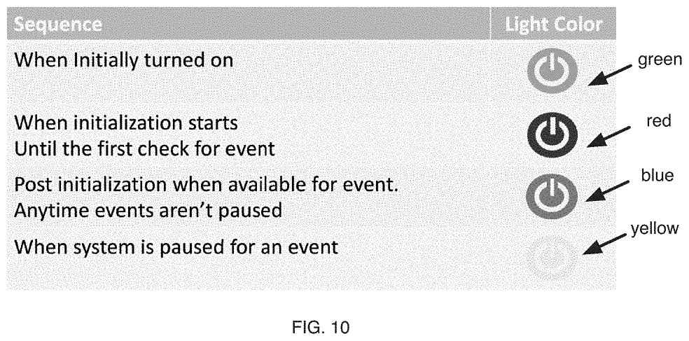

A dog device 205, dog device attachment 206, and/or suitable components of embodiments of the system 200 can include any suitable number and/or type of physical input receiving components (e.g., buttons; etc.), which can function to collect physical inputs from one or more users. Physical input receiving components preferably facilitate initialization and turning off of a dog device 205 and/or dog device attachment 206, but can additionally or alternatively trigger, perform, and/or be associated with any suitable functionality (e.g., of embodiments of the method 100, etc.). Physical input receiving components preferably indicate (e.g., through light color; etc.) one or more statuses, such as shown in FIG. 10, but can additionally or alternatively indicate any suitable information. However, physical input receiving components can be configure din any suitable manner.

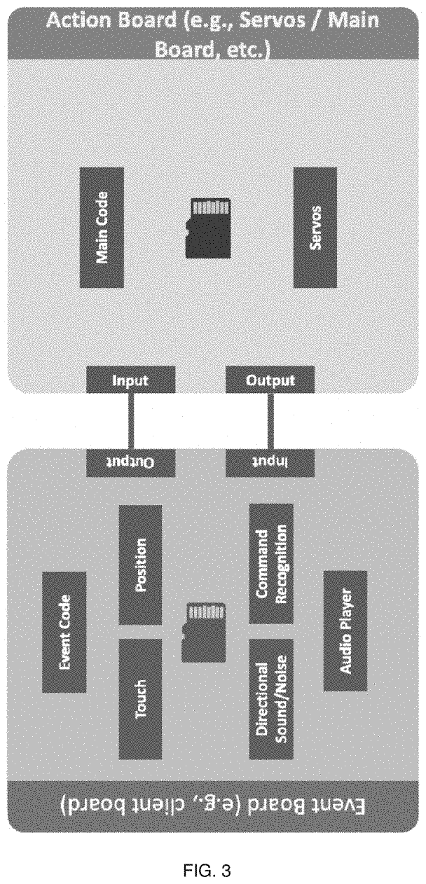

A dog device 205, dog device attachment 206, and/or suitable components of embodiments of the system 200 can include any suitable number and/or type of computing systems (e.g., including one or more processors, boards, storage components, etc.), which can be positioned at any suitable location and/or oriented in any suitable manner. In a specific example, computer processing systems 220 (e.g., including one or more boards and/or servers can perform functionality (e.g., distribution of functionality; etc.) shown in FIG. 3 and/or FIG. 11.

The dog device 205 preferably includes a computer processing system 220 including any suitable number of components. Computer processing associated with the dog device 205 can be performed by any suitable number of computer processing systems 220 including any number of boards, servers (e.g., control servers, event servers, etc.). In a specific example, the computer processing system 220 of the dog device 205 includes a single piece of hardware. In a specific example, the computer processing system 220 of the dog device 205 includes multiple pieces of hardware (e.g., two boards, etc.).

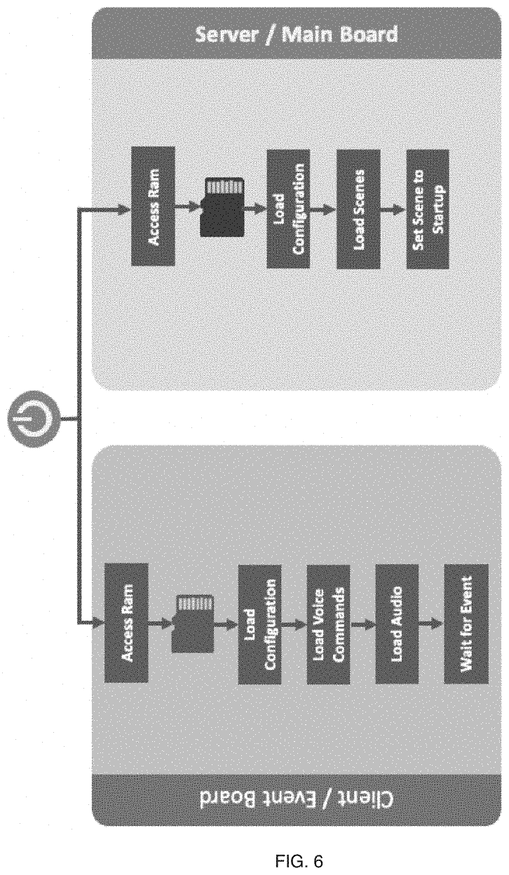

In a specific example, boards can perform functionality as shown in FIG. 6 for when a dog device 205 is initialized (e.g., by a user pressing a physical input receiving component such as an initialization button; etc.).

Computing systems can include any suitable storage components (e.g., RAM, direct-access data storage, etc.). In examples, configurations, scenes (e.g., scene files; scene parameters; etc.), event-related data, audio data (e.g., types of audio outputs; audio files; etc.), output action parameters, and/or any suitable data can be stored at one or more storage components. Scene parameters (e.g., mechanical output component parameters such as servos parameters, for operating mechanical output components; audio output component parameters; etc.) and/or suitable output action parameters can be captured and recorded from human operators (e.g., puppeteers; etc.) of output action components of the dog device 205, such as through recording signals (e.g., with a signal receiver; etc.) from the human operation. Additionally or alternatively, storage components and/or associated data can be configured in any suitable manner. However, computing systems can be configured in any suitable manner.

Embodiments of the system 200 can include one or more dog device attachments 206 (e.g., a base, emulating the appearance of a blanket and/or dog bed; attachments physically and/or wirelessly connectable to any suitable regions of the dog device 205; etc.). Dog device attachments 206 can charge the dog device 205 (e.g., wired charging; wireless charging such as inductive wireless charging with a battery coil positioned at the stomach region and/or other suitable region of a dog device 205; etc.), communicate with the dog device 205 (e.g., for performing system updates; for receiving and/or transmitting data; etc.), and/or performing any suitable functionality associated with embodiments of the method 100. In a specific example the system 200 can include a dog device attachment 206 shaped to fit the base of the dog device 205 (e.g., where the dog device attachment 206 can act as a base, such as a base emulating the appearance of a blanket and/or dog bed; etc.), where the dog device attachment 206 includes a charging component for charging the dog device 205. However, dog device attachments 206 can be configured in any suitable manner.

In a specific example, the system 200 can include a dog device including: a set of sensors for receiving inputs from a user; a processing system for: determining an event based on the inputs, the event comprising at least one of a touch event, a voice command recognition event, and a dog device position event; and processing a scene based on the event, the scene including scene parameters indicating instructions for a first output action; and a set of mechanical actuators and at least one speaker, for performing an output action based on the scene, wherein the output action comprises at least one of a mechanical output action and an audio output action.

However, embodiments of the system 200 can be configured in any suitable manner.

### 4. Other.

Embodiments of the method 100 and/or system 200 can include every combination and permutation of the various system components and the various method processes, including any variants (e.g., embodiments, variations, examples, specific examples, figures, etc.), where portions of embodiments of the method 100 and/or processes described herein can be performed asynchronously (e.g., sequentially), concurrently (e.g., in parallel), or in any other suitable order by and/or using one or more instances, elements, components of, and/or other aspects of the system 200 and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the method 100 and/or system 200 can be embodied and/or implemented at least in part as a machine configured to receive a computer-readable medium storing computer-readable instructions. The instructions can be executed by computer-executable components that can be integrated with embodiments of the system 200. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the method 100, system 200, and/or variants without departing from the scope defined in the claims. Variants described herein not meant to be restrictive. Certain features included in the drawings may be exaggerated in size, and other features may be omitted for clarity and should not be restrictive. The figures are not necessarily to scale. Section titles herein are used for organizational convenience and are not meant to be restrictive. The description of any variant is not necessarily limited to any section of this specification.

We claim:

1. A method for operating a dog device, comprising:
receiving a first input, at a sensor of the dog device, from a user;
determining an event based on the first input, the event comprising at least one of a touch event, a voice command recognition event, and a dog device position event;
processing a scene based on the event, the scene including scene parameters indicating instructions for a first output action; and
causing the dog device to perform the first output action based on the scene, wherein the first output action comprises at least one of a mechanical output action and an audio output action.

2. The method of claim 1, wherein the first input comprises sensor input data comprising at least one of: touch sensor data, audio sensor data, light sensor data, mechanical actuator sensor data, and biometric sensor data.

3. The method of claim 2, wherein the sensor input data comprises light sensor data, and wherein processing the scene comprises determining a scene associated with a low activity level for the first output action, based on the light sensor data.

4. The method of claim 1, wherein the sensor of the dog device comprises a touch sensor, wherein the event comprises a petting event comprising at least one of a slow petting event and a fast petting event, wherein determining the event comprises determining the petting event based on a set of touch events received at the touch sensor over a time period, wherein processing the scene comprises determining the scene based on the petting event.

5. The method of claim 1, wherein the sensor of the dog device comprises an audio sensor, wherein the event comprises a voice command recognition event, wherein determining the event based on the first input comprises determining the voice command recognition event based on an audio input received at the audio sensor of the dog device, wherein processing the scene comprises determining the scene based on the voice command recognition event, and wherein causing the dog to perform the first output action comprises causing the dog to simultaneously perform the mechanical output action and the audio output action based on the scene.

6. The method of claim 1, further comprising:
monitoring for a second input at a set of sensors of the dog device, the set of sensors comprising the sensor;
determining a lack of the second input after a predetermined time period threshold; and
determining a sleep scene based on the lack of the second input.

7. The method of claim 1, where processing the scene comprises determining the scene associated with a scene type from a set of scene types comprising at least one of: waking up scene types, sleep scene types, touch scene types, petting scene types, position scene types, speak scene types, howl scene types, hush scene types, excited scene types, and movement scene types.

8. The method of claim 1, wherein the dog device comprises a set of mechanical actuators comprising a set of mechanical actuator sensors, wherein causing the dog device to perform the first output action comprises causing the dog device to perform the first output action with the mechanical actuators based on the scene, and wherein the method further comprises:
receiving mechanical actuator sensor data during the performance of the first output action by the dog device;
determining a status of the performance of the first output action by the dog device based on the mechanical actuator sensor data; and
causing the dog device to perform a second output action based on the status of the performance of the first output action by the dog device.

9. The method of claim 8, wherein determining a status of the performance of the first output action by the dog device comprises determining a status of the performance of the first output action by the dog device based on the mechanical actuator sensor data during performance of the scene by the dog device, and wherein causing the dog device to perform the second output action comprises causing the dog device to perform a modified version of the first output action for completion of the scene.

10. The method of claim 8, wherein determining the status of the performance of the first output action and causing the dog device to perform the second output action are for facilitating improvement of safety of the user and the dog device.

11. The method of claim 8, further comprising determining strain and temperature associated with the set of mechanical actuator sensors based on the mechanical actuator sensor data, wherein the strain and temperature are associated with the performance of the first output action, and wherein determining the status of the performance of the first output action by the dog device comprises determining the status of the performance of the first output action based on the strain and temperature associated with the set of mechanical actuator sensors.

12. The method of claim 1, wherein causing the dog device to perform the first output action comprises causing the dog device to perform the first output action based on the scene, for facilitating improvement of a mental condition of the user, the mental condition comprising at least one of: dementia, Alzheimer's, depression, anxiety, psychosis, bipolar disorder, ADD, ADHD, and autism spectrum disorder.

13. The method of claim 12, wherein causing the dog device to perform the first output action comprises causing the dog device to perform the first output action based on the scene, for facilitating improvement of the mental condition through facilitating production of oxytocin in the user.

14. A system comprising a dog device comprising:
a set of sensors for receiving inputs from a user;
a processing system for:
determining an event based on the inputs, the event comprising at least one of a touch event, a voice command recognition event, and a dog device position event; and
processing a scene based on the event, the scene including scene parameters indicating instructions for a first output action; and
a set of mechanical actuators and at least one speaker, for performing an output action based on the scene, wherein the output action comprises at least one of a mechanical output action and an audio output action.

15. The system of claim 14, wherein the set of sensors of the dog device comprises: at least one touch sensor and at least one audio sensor.

16. The system of claim 15, wherein the set of sensors of the dog device further comprises at least one mechanical actuator sensor for receiving mechanical actuator sensor data, wherein the processing system is operable to determine updated scene parameters based on the mechanical actuator sensor data.

17. The system of claim 16, wherein the set of sensors of the dog device further comprises at least one light sensor for receiving light sensor data, wherein the processing system is operable to determine the scene based on the light sensor data.

18. The system of claim 17, wherein the set of sensors of the dog device further comprises at least one biometric sensor for collecting medical-related data from the user for characterizing at least one of: heart arrhythmia, heart rate variation, blood pressure, respirations, temperature, blood oxygen levels, blood glucose levels, sepsis detection, seizures, stroke, fall detection, and sleep monitoring.

19. The system of claim 14, further comprising a dog device attachment shaped to fit the base of the dog device, wherein the dog device attachment comprises a charging component for charging the dog device.

20. The system of claim 14, wherein the set of sensors, the processing system, and the set of mechanical actuators and the at least one speaker are for facilitating improvement of a mental condition of the user through facilitating production of oxytocin in the user, the mental condition comprising at least one of: dementia, Alzheimer's, depression, anxiety, psychosis, bipolar disorder, ADD, ADHD, and autism spectrum disorder.
